(19) United States
(12) Patent Application Publication
(10) Pub. No.: US 2007/0167838 A1
(43) Pub. Date: Jul. 19, 2007

(54) System and method for a Raman and/or fluorescence colposcope

(76) Inventors: Hugh W. Hubble, Swissvale, PA (US); Jeffrey K. Cohen, Pittsburgh, PA (US)

Correspondence Address: DUANE MORRIS LLP, 1667 K. STREET, N.W., SUITE 700, WASHINGTON, DC, 20006-1608, US

(21) Appl. No.: 11/595,977

(22) Filed: Nov. 13, 2006

Related U.S. Application Data
(60) Provisional application No. 60/735,319, filed on Nov. 10, 2005.

(51) Int. Cl. A61B 1/06; A61B 6/00

(52) U.S. Cl. 600/476; 600/167

(57) ABSTRACT

A colposcope and a method of using a colposcope which integrates both visual imaging capability and Raman imaging and/or fluorescence imaging is disclosed. In an embodiment, two sets of optics may be positioned within the housing of a colposcope to allow for both visual and Raman imaging. A Raman data set may be produced which may include a Raman image or a Raman spectrum of a cell, tissue, or a cancer cell, for example. Additionally, the use of one or more lasers for imaging and/or treatment is disclosed. A Raman imaging colposcope according to one embodiment of the present disclosure may be used to identify a cancer cell in vivo, giving a physician a tool to diagnose cervical cancer in his office. This instrument would also be of low cost and easy to operate.

### RELATED APPLICATIONS

The present application hereby incorporates by reference in its entirety and claims priority benefit from U.S. Provisional patent application Ser. No. 60/735,319 filed 10 Nov. 2005 titled “Raman and/or Fluorescence Colposcope”.

### BACKGROUND

A colposcope is a magnifying instrument used to examine the vagina and cervix. Abnormal cells may be identified and collected for analysis in vitro. A colposcope basically functions as a lighted microscope, which may be binocular. The colposcope typically is used to magnify the view of the cervix, vagina and vulvar surface and may be used as an aid to visually identify abnormal tissue, such as cancerous tissue. Prior art colposcopes may utilize different magnification levels, such as a low magnification setting (2× to 6×) for observing a wide field of view, a medium magnification setting (8× to 15×) for observing a somewhat limited field of view, and a high magnification setting (15× to 25×) for detailed observation of a particular area of interest.

Prior art colposcopes are typically limited to viewing in the optical wavelength range (i.e., approximately 400 nm to 700 nm) and have one set of optics (e.g., lenses) to support the optical wavelength viewing. Certain prior art colposcopes may include the functionality of fluorescence imaging. However, the ability to obtain a Raman image and/or a Raman spectrum of a sample using a colposcope is lacking. Raman imaging is extremely useful in finding and identifying abnormal tissue and cells, such as cancer cells and pre-cancerous cells. Additionally, there is a need for a colposcope and method of using a colposcope that integrates both the visual imaging capability with Raman imaging and/or fluorescence imaging.

### BRIEF DESCRIPTION OF THE DRAWINGS

FIG. 1 is a representation of a conventional colposcope.

FIG. 2 is a schematic diagram of a colposcope according to an embodiment of the present disclosure having laser photon source, a monochromator and a charge-coupled device.

FIG. 3 is a schematic diagram of a colposcope according to an embodiment of the present disclosure having a laser photon source, an imaging spectrometer and a charge-coupled device.

FIG. 4 is a schematic diagram of a colposcope according to an embodiment of the present disclosure having a laser photon source, a monochromator with a charge-coupled device and an imaging spectrometer with a charge-coupled device.

FIG. 5 is a schematic diagram of a colposcope according to an embodiment of the present disclosure having two laser photon sources, a monochromator with a charge-coupled device and an imaging spectrometer with a charge-coupled device.

FIG. 6 is a graph that illustrates Raman spectrum of a cervical cancer tissue in comparison with other tissues.

FIG. 7 is a flow chart illustrating a method of operating a colposcope according to an embodiment of the disclosure.

### DETAILED DESCRIPTION

A colposcope and a method of using a colposcope which integrates both visual imaging capability and Raman imaging and/or fluorescence imaging is disclosed. In an embodiment, two sets of optics may be positioned within the housing of a colposcope to allow for both visual and Raman imaging. A Raman data set may be produced which may include a Raman image or a Raman spectrum of a cell, tissue, or a cancer cell, for example. Additionally, the use of one or more lasers for imaging and/or treatment is disclosed. A Raman imaging colposcope according to one embodiment of the present disclosure may be used to identify a cancer cell in vivo, giving a physician a tool to diagnose cervical cancer in his office. This instrument would also be of low cost and easy to operate.

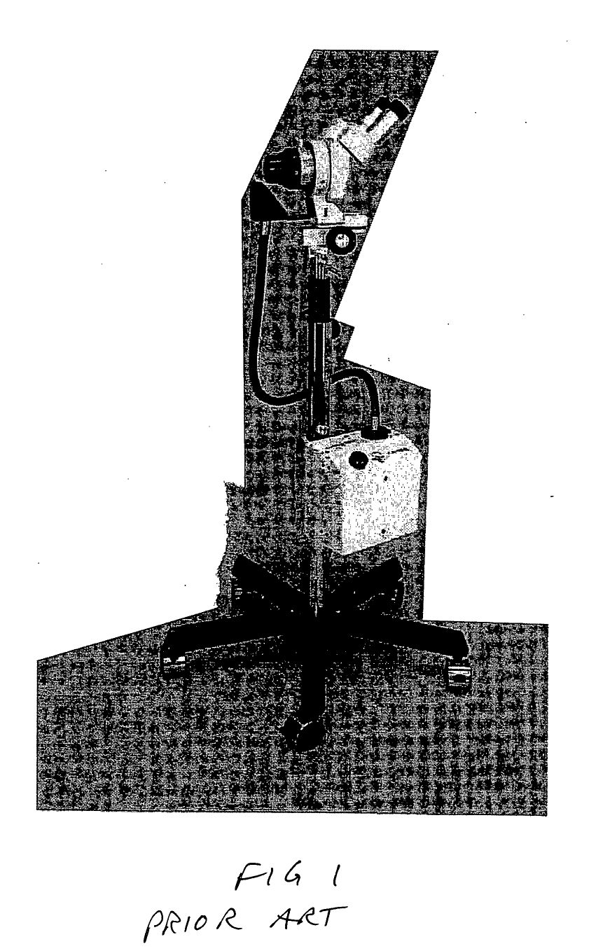

With attention directed toward FIG. 1, a conventional prior art colposcope is pictured. A colposcope typically functions as a lighted binocular microscope and may be used to magnify the view of the cervix, vagina and vulvar surface. A colposcope may be used as an aid to visually identify abnormal tissue, such as cancerous tissue.

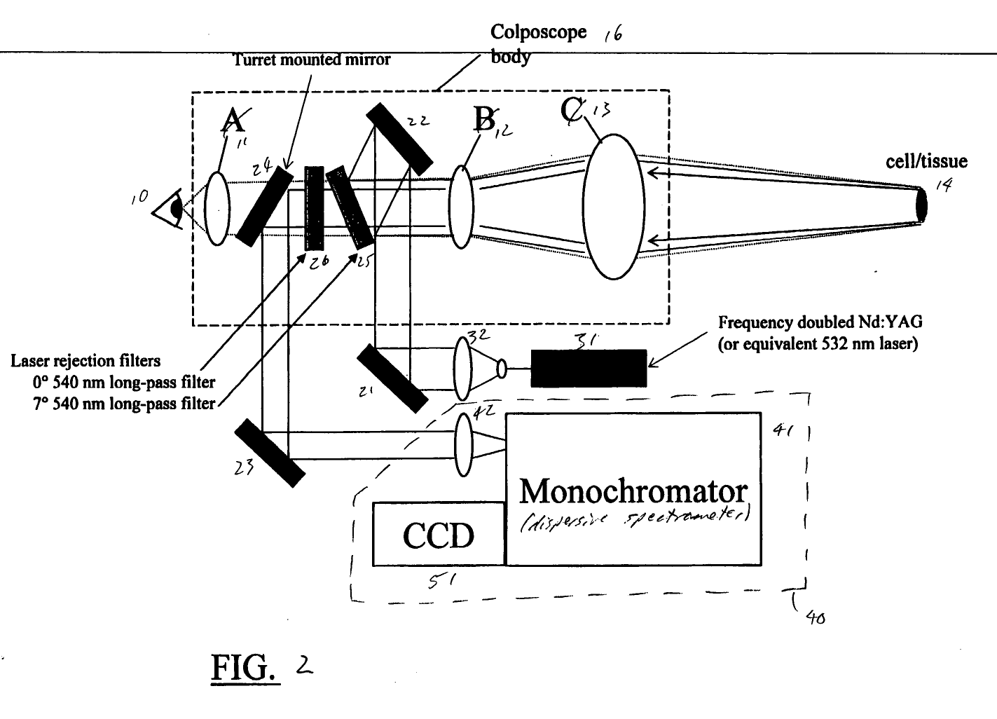

FIGS. 2 through 5 are each a schematic diagram of a colposcope according to an exemplary embodiment of the present disclosure where like reference numerals refer to like features throughout the Figures. With reference now to FIG. 2, an observer 10 may look through a first set of optics contained within a housing 16, sometimes referred to as a colposcope body. The first set of optics may include a lens 11, a lens 12, and a lens 13 which are optically coupled in order for the observer to view a sample 14. The sample 14 may be a cell, tissue, pre-cancerous cell, cancerous cell, or other similar object. A second set of optics may also be contained within the housing 16 and optically coupled to at least a part of the first set of optics. The second set of optics may include mirrors 21, 22, and 23, a rotatable mirror 24, a dichroic mirror 25, and a filter 26. As would be obvious to those of skill in the art, some of the mirrors, e.g., mirrors 21 and 23, are not necessary to practice the present disclosure. A photon source 31, which may preferably be a laser, and may also preferably be a laser emitting photons having a wavelength of approximately 532 nanometers, may be disposed so as to illuminate the sample with first photons so as to produce second photons. The photon source may preferably be mounted outside of the housing 16. The first photons may optionally pass through lenses 32 and may illuminate the sample via a portion of the second set of optics. As shown in an exemplary embodiment in FIG. 2, the first photons may reflect off of the mirrors 21 and 22, the dichroic mirror 25, and pass through the lenses 12 and 13 in order to reach the sample 14. As would be obvious to those of skill in the art, other possible arrangements of mirrors/lenses are contemplated while keeping to the principles of the disclosure. The second photons may be produced by the interaction of the first photons and the sample and the second photons may pass through the colposcope to be received by a photon detector module 40 which may include a monochromator (e.g., a dispersive spectrometer) 41 and be detected by a charge-coupled device 51 in order to produce a Raman scatter data set of the sample 14. Optionally, the second photons may pass through lens 42 prior to entering the monochromator. The Raman scatter data set may include, for example, a Raman image, a Raman spectrum, or, alternatively, a fluorescent image where the second photons are produced by fluorescence caused by the interaction of the first photons with the sample. The second photons may pass through the lenses 13 and 12, the dichroic mirror 25, the filter 26, and the mirrors 24 and 23. However, it would be obvious to those of skill in the art that other useful arrangements of optics are contemplated for providing the second photons to the photon detector module 40.

The rotatable mirror 24 may be a turret-mounted mirror or other similarly-mounted mirror which allows for movement of the mirror out of the visual optic path of the observer 10. The filter 26, which may comprise more than one filter, is preferably a laser rejection filter. In a preferred embodiment, the laser 31 may emit photons having a wavelength of approximately 532 nm and the filter 26 may be a 540 nm long pass filter.

It is to be understood by those of skill in the art that a standard optical colposcope is a low magnification microscope with a long working distance. The lenses 11 (which may be referred to herein as an “eyepiece”), 12, and 13 may represent the optical lenses present in a standard colposcope. By inserting Raman illumination optics (e.g., the second set of optics described above) between the eyepiece and the imaging optics (e.g., lenses 12 and/or 13) of a standard colposcope, the standard colposcope design may be modified to inject a laser beam (e.g., the first photons) into the optical axis of the colposcope. An example of the optics that may be inserted into a standard colposcope to convert it into a Raman imaging colposcope may include a portion of the optics for the Raman Illuminator system designed by the ChemImage Corporation of Pittsburgh, Pa.

In one embodiment, laser light (e.g., the first photons) from the photon source (e.g., the laser source shown below the colposcope body 16 in FIG. 2) may illuminate the target tissue (e.g., sample 14). This illumination of the target tissue by the first photons is not possible in a standard prior art colposcope without the modification of at least the second set of optics taught by the present disclosure. Where the second set of optics are inserted into the colposcope body 16 and configured to direct the first and second photons as described above, the photon detector module 40 may receive the second photons to produce a Raman scatter data set. The rotatable mirror 24, when positioned to redirect the second photons in FIG. 2 to the photon detector module 40, along with the filter 26 protect the observer 10 from eye damage from laser light exposure. Thus, Raman images and/or Raman spectra of the sample 14 can be obtained in vivo without the need to topically apply any optically active contrast agents (e.g., fluorescent dyes or quantum dots) to areas of tissue at risk in order to monitor the cell biomarkers or to obtain an image of the cell at risk.

The monochromator 41 may include a Fiber Array Spectral Translator (“FAST”). The FAST system can provide rapid real-time analysis for quick detection, classification, identification, and visualization of the sample. FAST technology can acquire a few to thousands of full spectral range, spatially resolved spectra simultaneously. This may be done by focusing a spectroscopic image onto a two-dimensional array of optical fibers that are drawn into a one-dimensional distal array with, for example, serpentine ordering. The one-dimensional fiber stack may be coupled to an imaging spectrograph of charge-coupled device, such as the charge-coupled device 51. One advantage of this type of apparatus over other spectroscopic apparatus is speed of analysis. A complete spectroscopic imaging data set can be acquired in the amount of time it takes to generate a single spectrum from a given material. FAST can be implemented with multiple detectors.

The FAST system allows for massively parallel acquisition of full-spectral images. A FAST fiber bundle may feed optical information from its two-dimensional non-linear imaging end (which can be in any non-linear configuration, e.g., circular, square, rectangular, etc.) to its one-dimensional linear distal end. The distal end feeds the optical information into associated detector rows. The detector may be the charge-coupled device 51 which has a fixed number of rows with each row having a predetermined number of pixels.

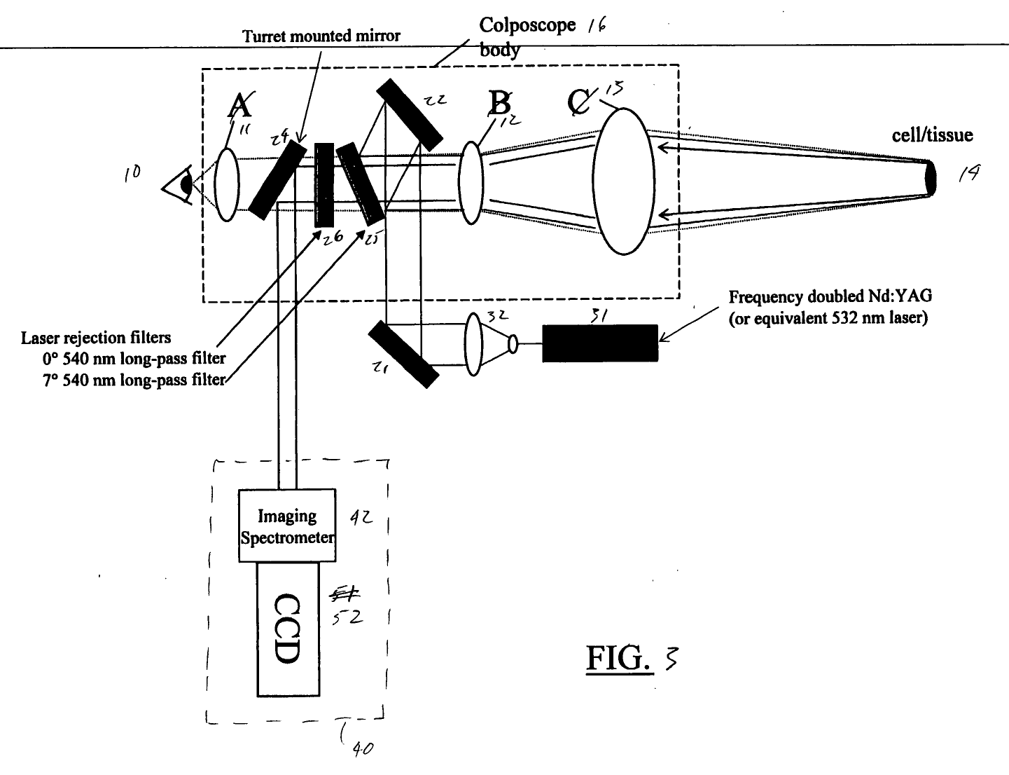

In the embodiment shown in FIG. 2, the photon detector module 40 comprises a monochromator 41 and a charge-coupled device 51. The difference between the FIG. 2 embodiment and the embodiment shown in FIG. 3, is that in FIG. 3 the photon detector module 40 comprises an imaging spectrometer 42 and a charge-coupled device 52. In one embodiment, the imaging spectrometer 42 may include a Liquid Crystal Tunable Filter (“LCTF”), as is known in the art. In addition to an LCTF-based spectrometer, some other examples of imaging spectrometers include FAST-based spectrometers and Computed Tomography Imaging Spectrometers. All other aspects of the embodiment in FIG. 3 are as described above for FIG. 2.

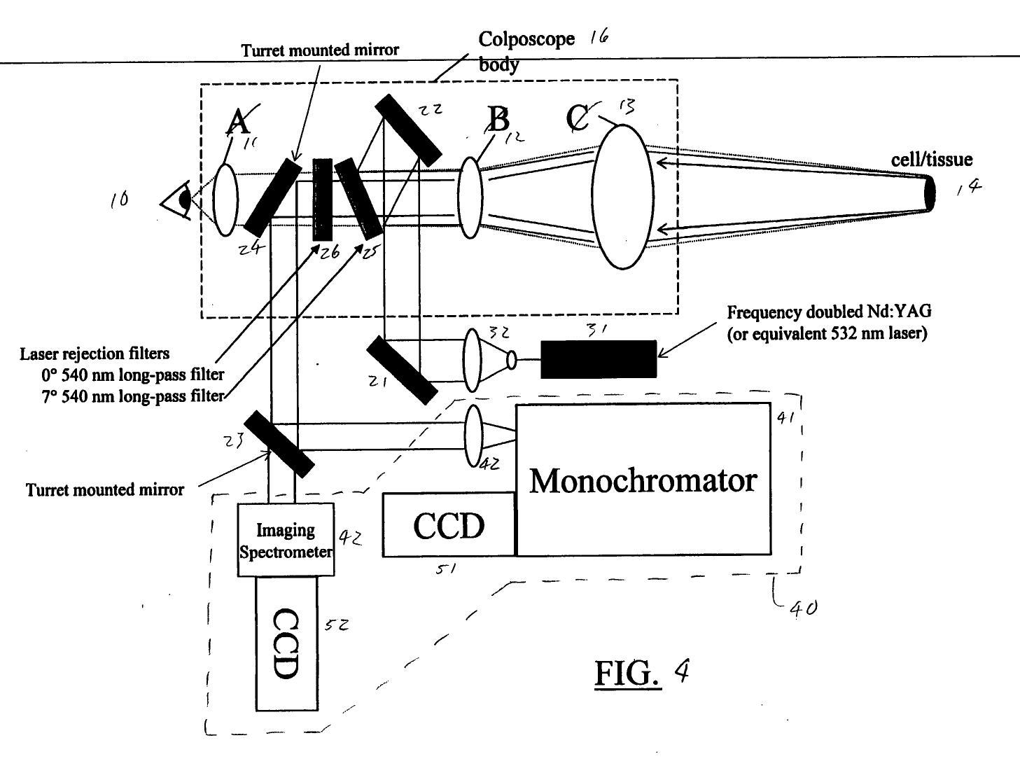

In the embodiment shown in FIG. 4, the photon detector module 40 includes both the monochromator 41 and a charge-coupled device 51 as shown in FIG. 2 and the imaging spectrometer 42 and a charge-coupled device 52 as shown in FIG. 3. Additionally, the mirror 23 is a rotatable mirror so as to direct the photons either to the monochromator 41 or the spectrometer 42. Those of skill in the art will readily recognize that other physical arrangements of the elements diagramed in FIG. 4 may be utilized without going beyond the scope of the present disclosure. All other aspects of the embodiment in FIG. 4 are as described above for FIG. 2.

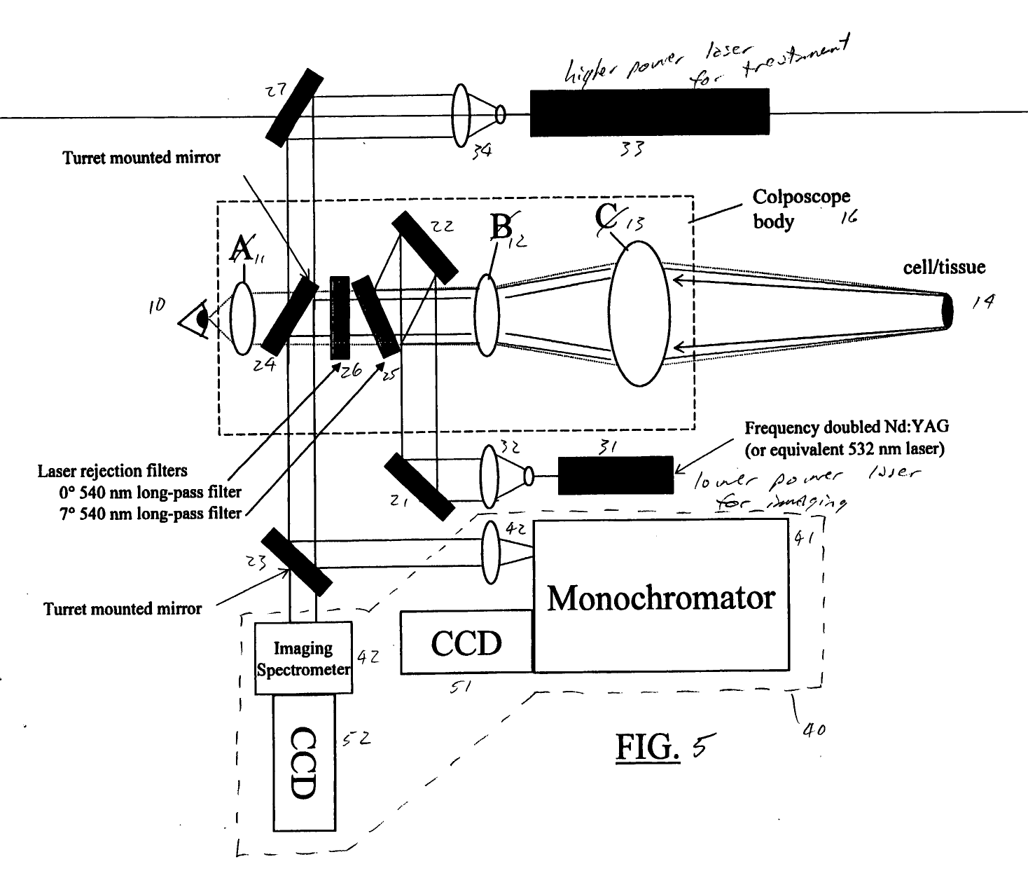

With attention now directed to FIG. 5, another embodiment of the present disclosure is depicted. The embodiment in FIG. 5 is the same as the embodiment depicted in FIG. 4 with the addition of a second photon source 33, optional lenses 34, and a mirror 27, which may be optional depending on the physical orientation of the second photon source with respect to the colposcope body 16, as would be obvious to those of skill in the art. Additionally, the rotatable mirror 24 is capable of directing third photons from the second photon source to the sample 14. Furthermore, the filter 26 may be displaced so as to not block the third photons from reaching the sample 14. The second photon source may preferably be a laser providing higher power laser light than the first photon source. The laser light from the second photon source (i.e., the third photons) are preferably used for treatment of the sample 14, e.g., when the sample is a pre-cancerous or cancerous cell, or other cell/tissue that may require laser treatment, such as a malignant cell. The laser light from the second photon source is typically not used for imaging or spectroscopy. All other aspects of the embodiment depicted in FIG. 5 are as described above for FIG. 4.

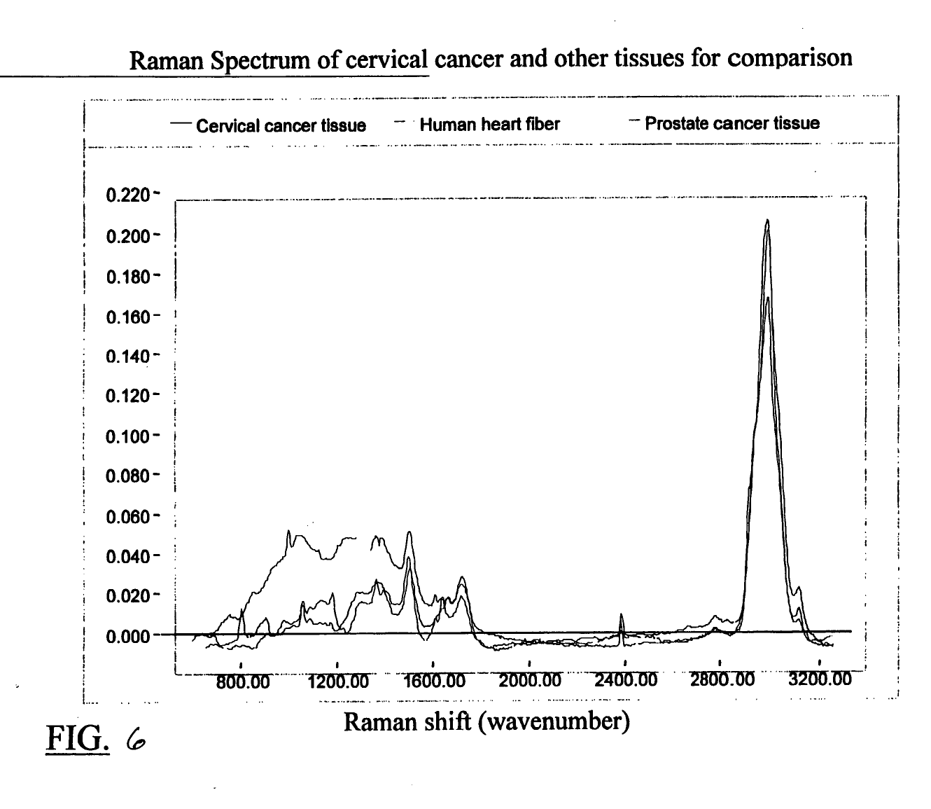

FIG. 6 illustrates a Raman spectrum of a cervical cancer tissue in comparison with a Raman spectrum from a human heart fiber and a prostate cancer tissue. While the spectra shown in FIG. 6 were not taken using a colposcope built according to the teachings of the present disclosure, the spectra are presented here to illustrate that a cervical cancer tissue may be a good candidate for observation of Raman scatter and, hence, a colposcope designed according to the teachings of the present disclosure may be configured to observe cervical and other cancer tissues through their Raman spectra and/or images.

In embodiments in which a fluorescence colposcope is used, the filter 26 in FIGS. 2 through 5 for a Raman colposcope may be modified or substituted with rejection filters designed to handle the wavelengths of a fluorescence light. In one embodiment of a colposcope in which both Raman and fluorescence is used, a larger bandwidth may be required of those laser rejection filters 26, as would be obvious to those of skill in the art. In one embodiment, the optics contained in a Falcon/Falcon II chemical imaging microscope developed by Chemlmage Corporation of Pittsburgh, Pa, may be suitably modified to obtain a colposcope design as embodied in FIGS. 2 through 5 and/or described above.

As low laser powers may be used for the first photon source 31 for use in live cell biological sample imaging, the first photon source may be very small in size and power since little more than a laser pointer is required. In one embodiment (not shown), the first photon source 31 laser could be built into the colposcope, eliminating the need for mirrors 21 and/or 22, for example, as well as eliminating any fiber optic laser delivery system.

In embodiments using a Raman colposcope, it may be possible to observe a sufficient number of key Raman lines identified as cancer markers without requiring broad spectral ranges and line-width limited spectral performance. This feature allows for a simpler colposcope design that allows for a faster on-site (i.e., at a doctor's site where the patient is present, as opposed to a remote laboratory site) and in vivo diagnosis of cancerous tissues/cells. The additional equipment needed (e.g., external laser sources, spectrometers, etc.) could be mounted to the side of the colposcope or on the base of the colposcope designed according to the teachings of the present disclosure.

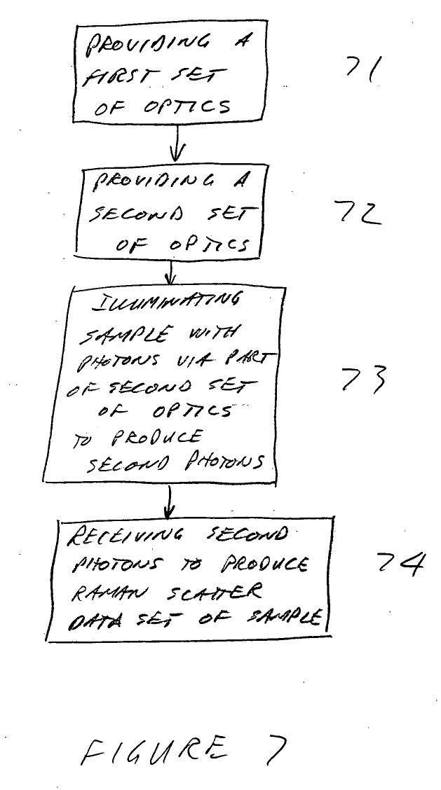

With reference now to FIG. 7, a flow chart indicating a method of using a colposcope according to the principles of the present disclosure is depicted. At step 71, a first set of optics is provided, preferably positioned within a housing, such as the housing 16 in FIG. 2. At step 72, a second set of optics is provided, preferably positioned within the housing 16 and optically coupled to at least a part of the first set of optics. At step 73, a sample, such as the sample 14 of FIG. 2, is illuminated with photons that travel via a portion of the second set of optics to interact with the sample 14 so as to produce second photons. At step 74, the second photons are received by, for example, the photon detector module 40 of FIG. 2 to thereby produce a Raman scatter data set of the sample 14.

The above description is not intended and should not be construed to be limited to the examples given but should be granted the full breadth of protection afforded by the appended claims and equivalents thereto. Although the disclosure is described using illustrative embodiments provided herein, it should be understood that the principles of the disclosure are not limited thereto and may include modification thereto and permutations thereof.

What is claimed is:

1. A colposcope comprising:
a housing;
a first set of optics positioned within said housing to enable a user to view an image of an in vivo sample; and
a second set of optics positioned within said housing and optically coupled to at least a part of said first set of optics,
a photon source for illuminating said sample with first photons via a portion of said second set of optics wherein said first photons interact with said sample to thereby produce second photons; and
a photon detector module for receiving said second photons to thereby produce a Raman scatter data set of said sample.

2. The colposcope of claim 1 wherein said image is an optical image.

3. The colposcope of claim 1 wherein said Raman scatter data set includes a Raman image.

4. The colposcope of claim 1 wherein said photon detector module receives said second photons to thereby produce a fluorescent image.

5. The colposcope of claim 1 wherein said Raman scatter data set is a Raman spectrum.

6. The colposcope of claim 1 wherein said photon source is a laser.

7. The colposcope of claim 6 wherein said first photons have a wavelength of approximately 532 nanometers.

8. The colposcope of claim 1 wherein said sample is a cell or tissue.

9. The colposcope of claim 1 wherein said sample is a cancer cell.

10. The colposcope of claim 1 wherein said second set of optics includes a rotatable mirror and a filter.

11. The colposcope of claim 1 wherein said photon detector module includes an imaging spectrometer.

12. The colposcope of claim 11 wherein said imaging spectrometer is a liquid crystal tunable filter.

13. The colposcope of claim 11 wherein said photon detector module includes a charge-coupled device.

14. The colposcope of claim 1 wherein said photon detector module includes a dispersive spectrometer.

15. The colposcope of claim 14 wherein said photon detector module includes a fiber array spectral translator.

16. The colposcope of claim 14 wherein said photon detector module includes a charge-coupled device.

17. The colposcope of claim 1 wherein said photon source is positioned within said housing.

18. The colposcope of claim 1 further comprising a second photon source optically coupled to said second set of optics.

19. The colposcope of claim 1 wherein said second photon source is a laser.

20. The colposcope of claim 19 wherein said laser is a treatment laser and provides third photons to said sample via a portion of said second set of optics.

21. The colposcope of claim 1 wherein said photon detector module includes a fiber array spectral translator.

22. A method for obtaining a Raman scatter data set of an in vivo sample using a colposcope comprising:
providing a first set of optics positioned within a housing of said colposcope to enable a user to view an image of the sample;
providing a second set of optics positioned within said housing and optically coupled to at least a part of the first set of optics;
illuminating the sample with photons via a portion of said second set of optics wherein said photons interact with the sample to thereby produce second photons; and
receiving the second photons to thereby produce a Raman scatter data set of the sample.

23. The method of claim 22 wherein the Raman scatter data set includes a Raman image.

24. The method of claim 22 wherein the Raman scatter data set includes a fluorescent image.

25. The method of claim 22 wherein the Raman scatter data set is a Raman spectrum.

26. In a colposcope having a housing and a first set of optics positioned within the housing to enable a user to view an image of an in vivo sample, the improvement comprising:
a second set of optics positioned within said housing and optically coupled to at least a part of said first set of optics,
a photon source for illuminating said sample with first photons via a portion of said second set of optics wherein said first photons interact with said sample to thereby produce second photons; and
a photon detector module for receiving said second photons to thereby produce a Raman scatter data set of said sample.

27. The colposcope of claim 26 wherein said image is an optical image.

28. The colposcope of claim 26 wherein said Raman scatter data set includes a Raman image.

29. The colposcope of claim 26 wherein said photon detector module receives said second photons to thereby produce a fluorescent image.

30. The colposcope of claim 26 wherein said Raman scatter data set is a Raman spectrum.

31. The colposcope of claim 26 wherein said photon source is a laser.

32. The colposcope of claim 31 wherein said first photons have a wavelength of approximately 532 nanometers.

33. The colposcope of claim 26 wherein said sample is a cell or tissue.

34. The colposcope of claim 26 wherein said sample is a cancer cell.

35. The colposcope of claim 26 wherein said second set of optics includes a rotatable mirror and a filter.

36. The colposcope of claim 26 wherein said photon detector module includes an imaging spectrometer.

37. The colposcope of claim 36 wherein said imaging spectrometer is a liquid crystal tunable filter.

38. The colposcope of claim 36 wherein said photon detector module includes a charge-coupled device.

39. The colposcope of claim 26 wherein said photon detector module includes a dispersive spectrometer.

40. The colposcope of claim 39 wherein said photon detector module includes a fiber array spectral translator.

41. The colposcope of claim 39 wherein said photon detector module includes a charge-coupled device.

42. The colposcope of claim 26 wherein said photon source is positioned within said housing.

43. The colposcope of claim 26 further comprising a second photon source optically coupled to said second set of optics.

44. The colposcope of claim 26 wherein said second photon source is a laser.

45. The colposcope of claim 44 wherein said laser is a treatment laser and provides third photons to said sample via a portion of said second set of optics.

46. The colposcope of claim 26 wherein said photon detector module includes a fiber array spectral translator.
